(19) United States
(12) Patent Application Publication
(10) Pub. No.: US 2008/0078392 A1
(43) Pub. Date: Apr. 3, 2008

(54) Breath detection system

(76) Inventors: Dana G. Pelletier, Ortonville, MI (US); Michael P. Chekal, Brighton, MI (US)

Correspondence Address: DELPHI TECHNOLOGIES, INC., M/C 480-410-202, PO BOX 5052, TROY, MI, 48007, US

(21) Appl. No.: 11/541,497

(22) Filed: Sep. 29, 2006

(51) Int. Cl. A61M 16/00

(52) U.S. Cl. 128/204.23; 128/204.21

(57) ABSTRACT

A breath detection system includes a conduit for gas delivery, e.g. oxygen. A pressure transducer is configured to: monitor a static gas pressure responsive to inhalation and exhalation, at a predetermined location of the conduit interior; and output a pressure signal related to the static gas pressure. A differentiator outputs a pressure change rate signal including a voltage level related to a time differential of the pressure signal. A comparator outputs a comparator signal including a comparator output voltage level related to a difference between the voltage level included in the pressure change rate signal and a detection threshold, where a predetermined comparator output voltage level range is indicative of the beginning of inhalation. A gas-providing apparatus provides a Mask signal feedback to temporarily reduce system sensitivity and also delivers a predetermined amount of the gas to the conduit in response to the comparator signal indicating that the voltage level is within the predetermined comparator output voltage level range.

### BACKGROUND

The present disclosure relates to breath detection, and more particularly to a system and methods for providing oxygen in response to breath detection.

Breath detection systems generally determine the start of a breath by measuring the pressure in a cannula disposed in a patient's nostrils. At the start of a breath, a rapid intake of air through the nose occurs wherein, by the Venturi effect, the relatively large flow of air passing the cannula opening creates a low pressure within the cannula tube. A typical pressure drop may be, for example, less than 1 in. H₂O.

A pressure transducer located at the opposite end of the cannula translates the pressure readings to voltage. Once the pressure drop extends beyond a predetermined threshold, the breath detector signals for oxygen delivery.

Since the pressure change resulting from inhalation is small, most breath detection systems utilize a high gain amplifier to read the signal. However, if the patient's breathing is shallow, even the amplified signal may not extend beyond the predetermined threshold, resulting in the patient not receiving oxygen delivery at the start of each breath.

Furthermore, a transducer signal may be noisy, requiring filtering to improve accuracy and readability. However, filtering, either in analog (i.e. via amplifiers and/or comparators) or digital (i.e. via analog-to-digital conversion and software), may add delay to the time from the start of inhalation until breath detection is confirmed. Reducing such delay is preferable due, at least in part, to the recognition that a majority of the volume inhaled during a breath is complete within the first 200 milliseconds of the start of inhalation.

As such, there is a need for a breath detection system that increases accuracy of breath detection and/or reduces the time to confirm breath detection.

### SUMMARY

A breath detection system is disclosed herein. The breath detection system includes a conduit having an interior and configured to deliver a gas including oxygen. A pressure transducer, in fluid communication with the interior of the conduit, is configured to: monitor a static gas pressure, the static gas pressure responsive to inhalation and exhalation, at a predetermined location of the conduit interior; and output a pressure signal related by a predetermined transfer function to the static gas pressure. A differentiator, in operative communication with the pressure transducer, is configured to output a pressure change rate signal including a voltage level related by a predetermined differentiator transfer function to a time differential of the pressure signal. The breath detection system further includes a comparator in operative communication with the differentiator, the comparator configured to output a comparator signal including a comparator output voltage level related by a predetermined comparator transfer function to a difference between the voltage level included in the pressure change rate signal and a detection threshold, where a predetermined comparator output voltage level range is indicative of the beginning of inhalation. A gas-providing apparatus, in operative communication with the comparator, is configured to provide a Mask signal to decrease sensitivity of the breath detection system, and to provide a predetermined amount of the gas to the conduit during a gas delivery phase, in response to the comparator signal indicating that the voltage level is within the predetermined comparator output voltage level range.

### BRIEF DESCRIPTION OF THE DRAWINGS

Objects, features and advantages of embodiments of the present disclosure will become apparent by reference to the following detailed description and drawings, in which like reference numerals correspond to similar, though not necessarily identical components. Reference numerals having a previously described function may not necessarily be described in connection with other drawings in which they appear.

FIG. 1 is a schematic view of an embodiment of electronic control of a breath detection system, including a differentiator;

FIG. 2 is a chart depicting an embodiment of a pressure signal (in Bar) with respect to time;

FIG. 3 is a chart depicting an embodiment of a pressure change rate signal (in Volts), for the pressure signal of FIG. 2, with respect to time;

FIG. 4 is a schematic view of the embodiment of the electronic control of FIG. 1, further including a comparator and two switches;

FIG. 5 is a schematic view of the embodiment of the electronic control of FIG. 1, further including a comparator and a microprocessor;

FIG. 6 is a chart depicting an embodiment of a cycle of the breath detection system;

FIG. 7 is a flow diagram depicting an embodiment of a method of providing a gas; and

FIG. 8 is a flow diagram depicting an embodiment of a method of providing a gas via a conduit.

### DETAILED DESCRIPTION

Embodiment(s) of the breath detection system (and method(s) of using the same) disclosed herein may advantageously be used to detect the start of an inhalation by monitoring a pressure change in a nasal cannula. Furthermore, embodiment(s) of the breath detection system provide for the administration of oxygen to a patient in response to the detection of inhalation. As such, embodiment(s) disclosed herein may be adapted to substantially minimize the delay between the start of inhalation and the confirmation of breath detection.

It is to be understood that “monitoring,” as used herein, may refer to direct monitoring or indirect monitoring. It is to be further understood that indirect monitoring may include monitoring a signal, such as a voltage, indicative of that which is ultimately being monitored.

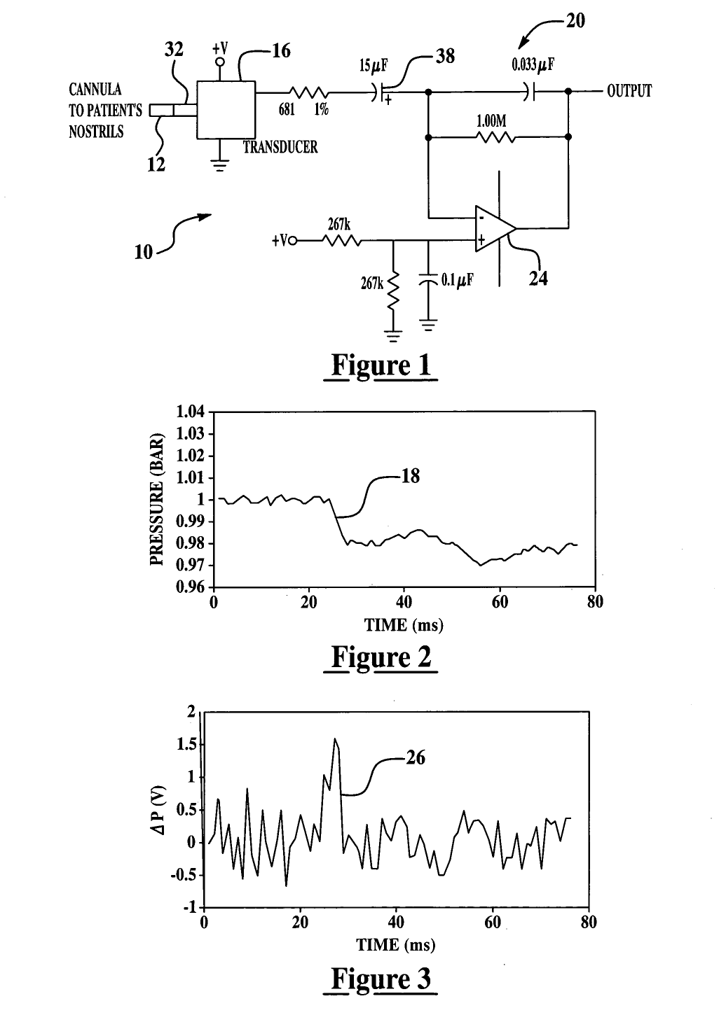

Referring now to FIG. 1, a schematic view of an embodiment of electronic control of a breath detection system 10 is illustrated. The breath detection system 10 of FIG. 1 depicts a conduit 12, which may include, for example, a nasal cannula and/or a face mask, in fluid communication with a pressure transducer 16. The conduit 12 is configured to deliver a gas. As a non-limitative example, the conduit 12 may be a cannula configured to deliver a gas (non-limiting examples of which include oxygen, an oxygen-containing gas, and/or the like) to a patient. Embodiment(s) of the present disclosure may advantageously be used with a conduit 12 having a single lumen at its gas-providing end (i.e. the end located nearest to the patient); whereas some prior breath detection devices generally require at least two lumens, one for delivering gas, and one for detecting breathing. As such, according to embodiment(s) herein, conduit 12 having a single lumen is configured to both detect breathing and deliver the gas via the single lumen.

The conduit 12, more specifically, the interior of conduit 12, may be in fluid communication with a pressure transducer 16. In an embodiment, the pressure transducer 16 is configured to monitor a static gas pressure at a predetermined location of the conduit 12 interior. It is to be understood that “static gas pressure” is defined as the potential pressure exerted in all directions by a fluid or gas at rest. For a fluid or gas in motion, static pressure is measured in a direction generally at right angles to the direction of flow.

The pressure transducer 16 may also be configured to produce and/or output a pressure signal 18. In an embodiment, the pressure transducer 16 is configured to translate the static gas pressure into a voltage, which may be included in the pressure signal 18.

As an example, FIG. 2 is a chart depicting an embodiment of a pressure signal 18 (in Bar) with respect to time. The pressure signal 18 may be indicative of the static gas pressure at the predetermined location of the conduit 12 interior. In an example, the pressure signal 18 is related by a predetermined transfer function to the static gas pressure at the predetermined location. In a non-limiting embodiment, the transfer function is substantially linear and extends through the origin, producing signals substantially directly proportional to static gas pressure.

Referring back to FIG. 1, it is to be understood that the static gas pressure is responsive to inhalation and exhalation. For example, when the conduit 12 is a nasal cannula positioned in a patient's nostrils, the fluid flow past the cannula during inhalation and exhalation creates low pressure within the conduit 12, in accordance with the Venturi effect, as mentioned above. The resulting pressure drop is often small, such as, for example, less than approximately 1 in. H₂O.

It is also to be understood that the predetermined location of the conduit 12 interior may refer to any area or point in the conduit where static gas pressure, which is responsive to inhalation and exhalation, may be monitored. As non-limitative examples, the predetermined location of the conduit 12 interior may be at the non-gas-providing end.

In an embodiment, the pressure transducer 16 may be in operative communication with a differentiator 20. The differentiator 20 may be a single Operational Amplifier (Op Amp) 24 configured as a high pass filter, with components added for stabilization. In an embodiment, the Op Amp 24 is operated with unipolar voltage rails, wherein its summing (+) node is set to an arbitrary common mode voltage, such as, for example, at the approximate midpoint between the rails.

In another embodiment, the differentiator 20 is configured to output a pressure change rate signal 26. The pressure change rate signal 26 may be embodied as a voltage level related, by a predetermined differentiator transfer function, to a time differential of the pressure signal 18. The pressure change rate signal 26 may be indicative of the rate of change of the pressure at the predetermined location in the conduit 12 interior. As such, the pressure signal 18 may be modified to generate the pressure change rate signal 26. As an example, the inputs of the differentiator transfer function may be the pressure signal 18 and time. It is to be understood that the time differential of the pressure signal 18 may be a linear function of pressure change rate. Other transfer functions may accomplish the same goal of amplifying pressure transducer signals that have a high rate of change, and attenuating pressure transducer signals that have a low rate of change.

As an example, FIG. 3 is a chart depicting an embodiment of a pressure change rate signal 26 (in Volts), for the pressure signal 18 of FIG. 2, with respect to time. In FIG. 3, it is to be recognized that the large spike, indicating a high rate of pressure change, between approximately 25 ms and 30 ms is associated with the relatively fast pressure drop depicted between approximately 25 ms and 30 ms in FIG. 2. As such, the activity between approximately 25 ms and 30 ms may be identified as a start of inhalation.

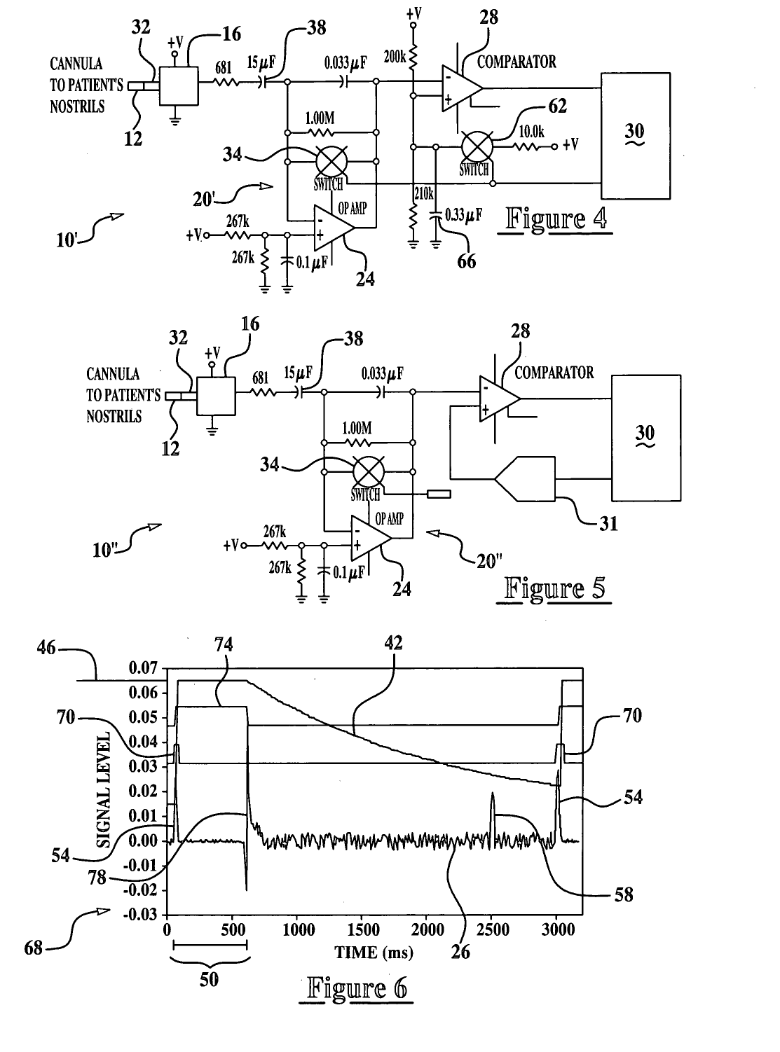

Referring now to FIGS. 1 and 4, in an embodiment of the breath detector 10′, differentiator 20 is in operative communication with a comparator 28, which is configured to output a comparator signal. The comparator signal may be embodied as a voltage level, related by a predetermined comparator transfer function to a difference between the voltage level of the pressure change rate signal 26 and a detection threshold. In an embodiment, the comparator transfer function is a step function with a transition at a threshold. It is to be understood that other transfer functions may accomplish the same goal of creating a type of switch that provides the information to the system 10 that inhalation has begun.

It is to be understood that the detection threshold may be a predetermined pressure at the predetermined location in the conduit 12, or a voltage associated therewith, and may be time dependent. The detection threshold is generally indicative of a beginning of inhalation. As such, the comparator 28 may be configured to detect the start of inhalation within milliseconds of its occurrence. As a non-limiting example, the comparator 28 may be configured to detect the start of inhalation within about 20 milliseconds of its occurrence. In an embodiment, the comparator 28 is configured to monitor the pressure change rate signal and to trip at a threshold that is slightly higher than the common mode voltage or detect when the pressure change rate signal fulfills a predetermined requirement.

Non-limitative examples of the predetermined requirement include reaching a predetermined comparator signal voltage range, or reaching (or extending beyond) a predetermined detection threshold. It is to be understood that the predetermined requirement may be indicative of the beginning of an inhalation. As such, detecting a pressure change rate signal 26 that has fulfilled the predetermined requirement may be associated with the beginning of inhalation. In an embodiment, a breath detection signal may be transmitted in response to the association with the beginning of inhalation.

The comparator 28 may also be in operative communication with a gas-providing apparatus 32. The gas-providing apparatus 32 may be configured to provide a predetermined amount of the gas to the conduit 12 during a gas delivery phase. In an embodiment, the gas-providing apparatus 32 is configured to provide the amount of gas in response to a comparator signal indicating that the voltage level has met the predetermined requirement. As a non-limitative example, the gas delivery phase may be less than about 500 ms. As another example, the gas delivery phase may range from about 250 ms to about 500 ms. In yet another example, the gas delivery phase may be of any duration less than the Mask phase (described further below).

In an embodiment, monitoring of the pressure change rate signal 26 may be ceased during a predetermined time period, also referred to herein as the “Mask phase.” The predetermined time period/Mask phase (described further hereinbelow with regard to reference numeral 50) includes the gas delivery phase, and may also include an amount of time before the gas delivery phase and/or an amount of time after the gas delivery phase. It is to be understood that “ceasing” is temporary. Further, temporarily ceasing monitoring the pressure change rate signal 26 may include: pausing monitoring of the pressure change rate signal 26 in response to detecting that the pressure change rate signal has fulfilled the predetermined requirement; providing the predetermined amount of gas to the conduit 12; and resuming monitoring of the pressure change rate signal 26.

In another embodiment, a gas delivery signal may be transmitted in response to detecting the beginning of inhalation. In yet another embodiment, the gas delivery signal may be transmitted in response to the breath detection signal. It is to be understood that pausing monitoring of the pressure change rate signal 26 and/or providing the predetermined amount of gas may be responsive to the gas delivery signal.

Ceasing, pausing, or masking the monitoring of the pressure change rate signal 26 may be responsive to pausing the monitoring of the pressure values embodied in the pressure signal 18. As such, pausing the monitoring of the pressure values may be responsive to the gas delivery signal. Similarly, monitoring the pressure values may be resumed following the gas delivery phase.

Referring now to FIG. 5, the gas-providing apparatus 32 may be in communication with a microcontroller 30. In an embodiment, the microcontroller 30 is configured to control a digital-to-analog converter (DAC) 31. In this embodiment, DAC 31 replaces switch 62 and capacitor 66 (shown in FIG. 4). The microcontroller 30 may also be adapted to sense the activation of the breath detection signal from comparator 28 and, in response thereto, provide the Mask signal to switch 34 (and to switch 62, e.g., in the embodiment of FIG. 4) and transmit the gas delivery signal; trigger the gas-providing apparatus 32 to provide the predetermined amount of gas; and cease transmission of the gas delivery signal following the gas delivery phase. In an embodiment, the gas delivery signal is transmitted in response to the predetermined comparator output signal indicating the beginning of inhalation.

Referring also again to FIG. 4, in an embodiment, the breath detection system 10′, 10″ includes a switch 34 in a feedback loop of the differentiator 20′, 20″. The switch 34 may be configured to select between differentiator 20′ and low gain modes for the amplifier 24 during a second predetermined time period, which may include the gas delivery phase. In an embodiment, the switch 34 is responsive to the gas delivery signal. As an example, the switch 34 may be configured to select the low gain amplifier in response to the gas delivery signal.

Referring now to FIG. 6, the detection threshold 42 may be increased to a predetermined level 46 during the mask phase 50. In an embodiment, the detection threshold 42 is increased to substantially prevent providing the gas at a time other than substantially at the beginning of inhalation 54. The predetermined level 46 may be any level at which it is substantially unlikely that the system will detect breathing activity.

In an embodiment, increasing the detection threshold 42 to the predetermined level 46 during the mask phase 50 may substantially prevent detecting “glitching” of the differentiator 20, 20′, 20″ and misidentifying it as the start of an inhalation. It is to be understood that “glitching,” as used herein, may occur when the switch 34 opens, and may include a brief spike in the Op Amp 24 output, which may occur as a result of a small bias on the input capacitor 38 that developed during the gas delivery phase and/or the gas delivery signal.

Referring still to FIG. 6, in another embodiment, increasing the detection threshold 42 to the predetermined level 46 during the mask phase 50 may substantially prevent the system 10, 10′, 10″ from detecting an end of exhalation 58 and misinterpreting it as the beginning of inhalation 54. The end of an exhalation 58 may have a similar dP/dT (pressure differential/time differential) waveform as the start of an inhalation 54, although it may be slightly smaller in value. If the system 10, 1′, 10″ detects the end of exhalation 58 and interprets it as the beginning of inhalation 54, the gas may be delivered at the end of exhalation 58, when it is less effective. Delivering the gas at the end of exhalation 58 may also result in preventing the subsequent beginning of inhalation 54 from being detected. As such, a system 10, 10′, 10″ configured to increase the detection threshold during the mask phase 50 may be better adapted to differentiate between a start of inhalation 54 and an end of exhalation 58.

Referring again to FIGS. 4 and 6, the breath detection system 10 may include a second switch 62. The second switch 62 may be adapted to operatively cause the detection threshold to increase in response to the gas delivery signal. In a non-limiting example, closure of switch 62 changes the divider ratio, bringing the threshold to the rail voltage, and deposits charge on capacitor 66, which charge is then slowly bled off after switch 62 opens, gradually reducing the threshold.

In an embodiment, the detection threshold 42 may decrease at a predetermined rate after the mask phase 50. In an embodiment, the detection threshold 42 decreases in response to the end of the gas delivery signal. It is to be understood that the predetermined rate may be of any form, including linear or exponential. As such, the sensitivity of the system 10, 10′, 10″ may start out relatively low after the mask phase 50 and may rise with time, whereby full sensitivity is delayed.

The breath detection system 10 may include a capacitor 66 (as shown in FIG. 4), which may be located on the voltage divider of the comparator 28. The capacitor 66 may be configured to operatively cause a relatively slow decrease in the detection threshold 42 at the predetermined rate. As a non-limiting example, “relatively slow” may refer to detection threshold 42 taking from about 200 milliseconds to about 1000 milliseconds to return to its pre-mask phase 50 level. In an embodiment, the capacitor 66 may have a value from about 0.33 microfarads to about 3.3 microfarads. In another embodiment, the capacitor may have a value from about 0.47 microfarads to about 2.2 microfarads.

Referring further to FIG. 6, in an example breath detection cycle 68, inhalation 54 occurs at 100 ms, wherein the pressure change rate signal 26 extends above the detection threshold 42, resulting in detection of the inhalation 54. The breath detection signal 70 is transmitted to the microcontroller 30 in response to detection of the inhalation, and microcontroller 30 responds by starting the gas delivery and issuing the Mask signal 74. The gas is delivered via the conduit 12 during the gas delivery phase, triggered by the breath detection signal 70. While the Mask signal 74 is active, the detection threshold 42 increases to a predetermined level 46, which is well above the pressure change rate signal 26, wherein the breath detection signal 70 essentially deactivates.

After the Mask phase 50, the Mask signal 74 deactivates. As the Op Amp 24 gain rises, the output briefly “glitches” 78. However, the “glitch” 78 is well below the detection threshold 42 and is, thus, ignored by the comparator 28.

Also after the Mask phase 50, the detection threshold 42 drops steadily at a predetermined rate. At 2500 ms, the end of an exhalation 58 occurs, causing a spike from the differentiator 20. The low amplitude of the exhalation 58 signal, coupled with the relatively high detection threshold 42 results in the exhalation 58 spike being ignored by the system. At 3000 ms, the start of an inhalation 54 occurs again. The relatively high amplitude of the signal 54, coupled with the relatively low detection threshold 42 results in detection of the inhalation 54 and issuance of the breath detection signal 70, whereby the cycle 68 repeats.

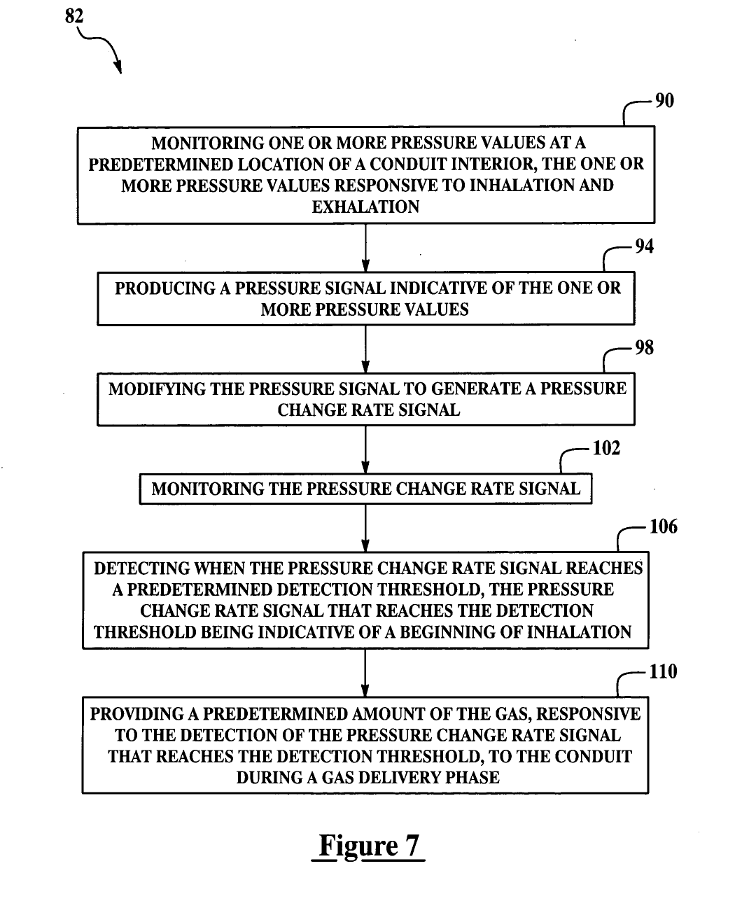

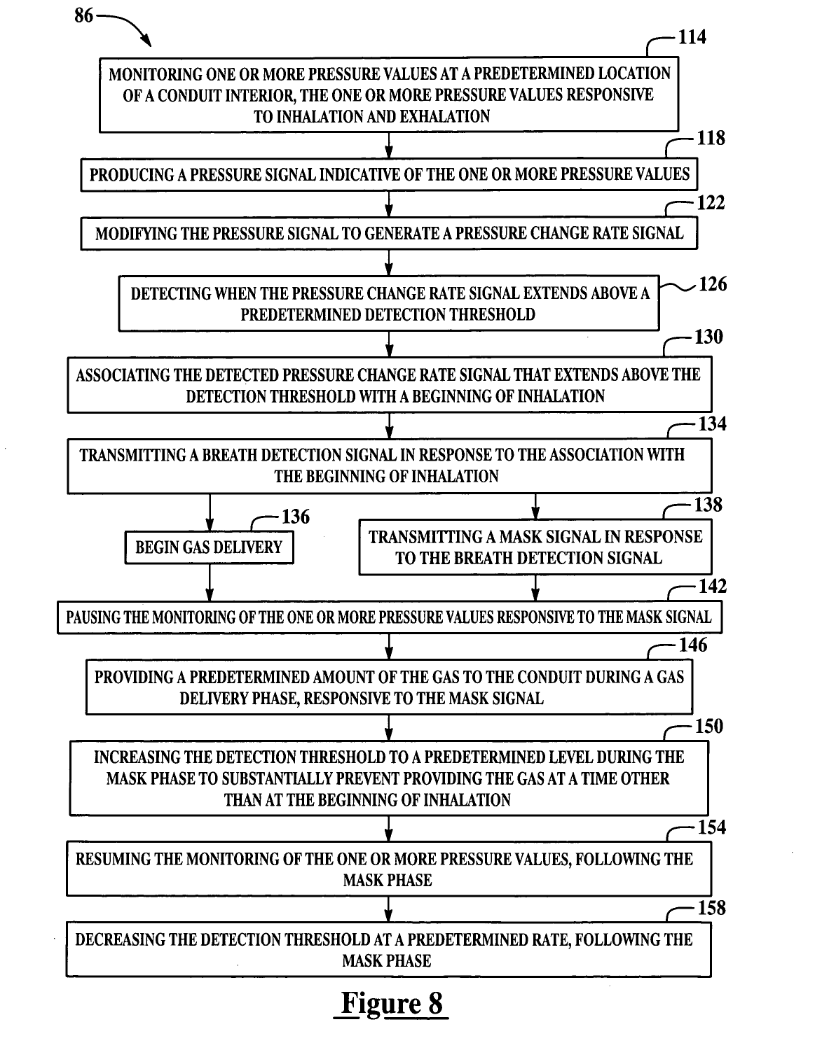

In accordance with the methods and system disclosed herein, FIG. 7 depicts an embodiment 82 of a method of providing an oxygen-containing gas, and FIG. 8 depicts a further embodiment 86 of the method.

Referring to FIG. 7, the embodiment of a method 82 of providing a gas includes monitoring one or more pressure values at a predetermined location of a conduit 12 interior, the one or more pressure values responsive to inhalation and exhalation, as depicted at reference numeral 90, and producing a pressure signal indicative of the one or more pressure values, as depicted at reference numeral 94. The method 82 may also include modifying the pressure signal to generate a pressure change rate signal, as depicted at reference numeral 98, and monitoring the pressure change rate signal, as depicted at reference numeral 102. Further, the method 82 may include detecting when the pressure change rate signal reaches a predetermined detection threshold, as depicted at reference numeral 106, and providing a predetermined amount of gas to the conduit 12 during a gas delivery phase, as depicted at reference numeral 110. In an embodiment, providing the predetermined amount of gas is responsive to the detection of the pressure change rate signal that reaches the detection threshold. It is to be understood that the pressure change rate signal that reaches the detection threshold may be indicative of a beginning of inhalation, as mentioned above.

Referring now to FIG. 8, an embodiment of a method 86 of providing a gas via a conduit 12 includes monitoring one or more pressure values at a predetermined location of the conduit 12 interior, as depicted at reference numeral 114, and producing a pressure signal indicative of the one or more pressure values, as depicted at reference numeral 118. The method 86 may also include modifying the pressure signal to generate a pressure change rate signal, as depicted at reference numeral 122, and detecting when the pressure change rate signal extends above a predetermined detection threshold, as depicted at reference numeral 126. Further, the method 86 may include associating the detected pressure change rate signal that extends above the detection threshold with a beginning of inhalation, as depicted at reference numeral 130; transmitting a breath detection signal in response to the association with the beginning of inhalation, as depicted at reference numeral 134; beginning gas delivery in response to the breath detection signal, as depicted at reference numeral 136; and transmitting a Mask signal in response to the breath detection signal, as depicted at reference numeral 138. Yet further, the method 86 may include pausing the monitoring of the one or more pressure values responsive to the Mask signal, as depicted at reference numeral 142, providing a predetermined amount of the gas to the conduit during a gas delivery phase responsive to the Mask signal, as depicted at 146, and increasing the detection threshold to a predetermined level during the Mask phase to substantially prevent providing the gas at a time other than substantially at the beginning of inhalation, as depicted at reference numeral 150. Even further, the method 86 may include resuming the monitoring of the one or more pressure values following the Mask phase, as depicted at reference numeral 154, and decreasing the detection threshold at a predetermined rate following the Mask phase, as depicted at reference numeral 158.

It is to be understood that the terms “communication,” “operative communication,” and/or the like are broadly defined herein to encompass a variety of divergent connected arrangements and assembly techniques. These arrangements and techniques include, but are not limited to (1) the direct communication between one component and another component with no intervening components therebetween; and (2) the communication of one component and another component with one or more components therebetween, provided that the one component being in “communication with” or “operative communication with” the other component is somehow ultimately connected with the other component (notwithstanding the presence of one or more additional components therebetween), by any means, such as, for example, electrically, fluidly, and/or physically. For example, the conduit 12 may be in communication with the differentiator 20 although the transducer 16 is disposed therebetween.

While several embodiments have been described in detail, it will be apparent to those skilled in the art that the disclosed embodiments may be modified. Therefore, the foregoing description is to be considered exemplary rather than limiting.

What is claimed is:

1. A breath detection system, comprising:
a conduit having an interior and configured to deliver a gas;
a pressure transducer in fluid communication with the interior of the conduit, the pressure transducer configured to monitor a static gas pressure at a predetermined location of the conduit interior, and output a pressure signal related by a predetermined transfer function to the static gas pressure, the static gas pressure being responsive to inhalation and exhalation;
a differentiator in operative communication with the pressure transducer and configured to output a pressure change rate signal including a voltage level related by a predetermined differentiator transfer function to a time differential of the pressure signal;
a comparator in operative communication with the differentiator, the comparator configured to output a comparator signal including a comparator output voltage level related by a predetermined comparator transfer function to a difference between the voltage level included in the pressure change rate signal and a detection threshold, a predetermined comparator output voltage level range being indicative of a beginning of inhalation; and
a gas-providing apparatus in operative communication with the comparator and configured to provide a Mask signal to decrease sensitivity of the breath detection system in response to the comparator signal indicating that the voltage level is within the predetermined comparator output voltage level range, the gas-providing apparatus further being configured to provide a predetermined amount of the gas to the conduit during a gas delivery phase in response to the comparator signal indicating that the voltage level is within the predetermined comparator output voltage level range.

2. The breath detection system of claim 1 wherein the conduit includes a single lumen at a gas-providing end of the conduit.

3. The breath detection system of claim 1, further comprising a switch in a feedback loop of the differentiator, the switch configured to select, responsive to a Mask signal, between the differentiator and a low gain amplifier during a second predetermined time period including the gas delivery phase.

4. The breath detection system of claim 3, further comprising a microcontroller in communication with the gas-providing apparatus, the microcontroller adapted to: in response to the predetermined comparator output voltage level range indicating the beginning of inhalation, transmit the Mask signal, in response to which the switch is configured to select the low gain amplifier; then trigger the gas-providing apparatus to provide the predetermined amount of the gas during the gas delivery phase; and cease transmission of the Mask signal following the gas delivery phase.

5. The breath detection system of claim 4 wherein, during the Mask phase, the detection threshold is increased to a predetermined level.

6. The breath detection system of claim 5, further comprising a second switch in communication with the comparator, the second switch adapted to operatively cause the detection threshold increase in response to the Mask signal.

7. The breath detection system of claim 5 wherein, after ceasing transmission of the Mask signal, the detection threshold decreases at a predetermined rate.

8. The breath detection system of claim 7, further comprising a capacitor adapted to operatively cause the decrease of the detection threshold at the predetermined rate.

9. The breath detection system of claim 8 wherein the capacitor is on a voltage divider of the comparator, the capacitor having a capacitor value ranging from about 0.33 microfarads to about 3.3 microfarads.

10. A method of providing an oxygen-containing gas, the method comprising:
monitoring one or more pressure values at a predetermined location of a conduit interior, the one or more pressure values responsive to inhalation and exhalation;
producing a pressure signal indicative of the one or more pressure values;
modifying the pressure signal to-generate a pressure change rate signal;
monitoring the pressure change rate signal;
detecting when the pressure change rate signal reaches a predetermined detection threshold, the pressure change rate signal reaching the detection threshold being indicative of a beginning of inhalation;
providing a Mask feedback signal to reduce sensitivity, responsive to the detection of the pressure change rate signal that reaches the detection threshold; and
providing a predetermined amount of the gas, responsive to the detection of the pressure change rate signal that reaches the detection threshold, to the conduit during a gas delivery phase.

11. The method of claim 10, further comprising temporarily ceasing monitoring of the pressure change rate signal during a predetermined time period including the gas delivery phase.

12. The method of claim 11 wherein the temporarily ceasing monitoring of the pressure change rate signal during the predetermined time period further comprises:
pausing monitoring of the pressure change rate signal in response to the detection of the pressure change rate signal that reaches the detection threshold;
providing the predetermined amount of the gas to the conduit in response to the detection of the pressure change rate signal that reaches the detection threshold; and
resuming monitoring of the pressure change rate signal after providing the predetermined amount of the gas to the conduit and waiting for the predetermined time period to expire.

13. The method of claim 10 wherein reduced sensitivity is achieved by increasing the detection threshold to a predetermined level while the Mask feedback signal is active.

14. The method of claim 13 wherein the detection threshold is increased to the predetermined level to substantially prevent providing the gas at a time other than substantially at the beginning of inhalation.

15. The method of claim 13, further comprising decreasing the detection threshold at a predetermined rate after the Mask feedback signal is deactivated.

16. The method of claim 15 wherein the predetermined rate delays detection of inhalation to substantially prevent detection of exhalation.

17. A method of providing an oxygen-containing gas-via a conduit having an interior, the method comprising:
monitoring one or more pressure values at a predetermined location of the conduit interior, the one or more pressure values responsive to inhalation and exhalation;
producing a pressure signal indicative of the one or more pressure values;
modifying the pressure signal to generate a pressure change rate signal;
detecting when the pressure change rate signal extends above a detection threshold;
associating the detected pressure change rate signal that extends above the detection threshold with a beginning of inhalation;
transmitting a breath detection signal in response to the association with the beginning of inhalation;
starting gas delivery and providing a predetermined amount of the gas to the conduit during a gas delivery phase, in response to the breath detection signal;
transmitting a Mask signal during a Mask phase, in response to the breath detection signal;
pausing the monitoring of the one or more pressure values, responsive to the Mask signal;
increasing the detection threshold to a predetermined level during the Mask phase to substantially prevent providing the gas at a time other than substantially at the beginning of inhalation;
resuming the monitoring of the one or more pressure values, following the Mask phase; and
decreasing the detection threshold at a predetermined rate, following the Mask phase.
